(19) United States
(12) Patent Application Publication
(10) Pub. No.: US 2008/0281268 A1
(43) Pub. Date: Nov. 13, 2008

(54) Portable Device for Heating Up Intravenous Fluids

(75) Inventor: Jan Erik Vest Hansen, Arhus C (DK)

Correspondence Address: CANTOR COLBURN, LLP, 20 Church Street, 22nd Floor, Hartford, CT, 06103, US

(73) Assignee: HOTWATCH APS, Arhus C (DK)

(21) Appl. No.: 12/091,831

(22) PCT Filed: Oct. 27, 2006
(86) PCT No.: PCT/DK2006/000598

§ 371 Date: Jun. 10, 2008

(30) Foreign Application Priority Data
Oct. 27, 2005 (EP) 050774694

(51) Int. Cl. A61M 5/44

(52) U.S. Cl. 604/114; 604/113

(57) ABSTRACT

A system is disclosed for heating a flow of intravenous fluid comprising an intravenous tube (3) for connecting a source of intravenous fluid to an intravenous catheter (9), the intravenous tube comprising heating means (21) integrated therein for raising the temperature of the intravenous flow in the tube and a portable unit (2) comprising the energy source for providing electrical power to the heating means integral with a pump (5) for controlling the flow of the fluid. Hereby is provided a path between a source of intravenous fluid and an intravenous catheter ensured to be highly hygienic, because the number of connections wherein the flow of intravenous fluid can be contaminated is limited to a minimum as compared to other systems with multiple connections to external units.

### FIELD OF INVENTION

The present invention relates to a system for heating a flow of intravenous fluid and to an assembly for a system for connecting a source of intravenous fluid to a patient.

### BACKGROUND

Army medics, paramedics, mountain rescue teams and other types of rescue personnel are often in need for administering intravenous fluids to critically wounded persons. However, when these intravenous fluids are carried in the field the temperature of these fluids is often substantially lower than the body temperature of the patient. The injection of cold intravenous fluids in to a vein of a patient presents a substantial risk for hyperthermia, which poses a threat to the patient's life. Therefore there is a need for a portable device to heat the intravenous fluid to a temperature closer to the body temperature before it enters the patient's veins.

WO 01/62194 A1 discloses a portable disposable intravenous fluid warming system. The system comprises a battery powered unit with an integrated heating element having an inlet and an outlet for intravenous fluids, sized to fit standard intravenous line connectors. The inlet of the unit is connected to an intravenous bag and the outlet is connected to an intravenous catheter via standard intravenous tubes.

U.S. Pat. No. 6,236,809 discloses a system for heating intravenous fluids comprising a disposable heat exchanger with an inlet and an outlet for intravenous fluids and a portable battery powered unit including a heating element and temperature controller. The inlet of the disposable heat exchanger comprises a fitting for connection to a source of intravenous fluid via a first length of tubing, and the outlet of the disposable heat exchanger likewise comprises a fitting for connection to an intravenous catheter via a second length of tubing.

U.S. Pat. No. 5,250,032 disclose yet another system for warming intravenous fluids. The system comprises a battery powered heating element and a control circuit placed in housing mounted on the patients arm. An elongated portion of a standard intravenous tube is inserted in a channel in the heating element, whereby a flow of intravenous fluid in the intravenous tube is heated

U.S. Pat. No. 3,908,652 disclose a medical infusion apparatus having an infusion flask exchangeably fastened to a casing, an infusion tube provided with a drip chamber, an electrical control system, and a drip valve or an infusion pump. A heat radiation source may be incorporated in the apparatus in order to heat the infusion liquid on its path from the flask to the infusion tube.

US 2002/0156451 A1, WO 96/11027 and EP 1 066 844 A1 all discloses heating means for heating of intravenous fluids by means of electrical resistor heaters arranged within the intravenous tube.

U.S. Pat. No. 4,684,367 discloses a system for intravenous delivery of fluids to patients, which includes means to apply a pressure to the bag holding the fluid and having fluid heating means, flow measuring means and flow regulating means that the flow of fluid passes before reaching the patient.

### BRIEF SUMMARY OF THE INVENTION

The invention provides a simple system to provide a heated flow of intravenous fluid prior to its entry into the vein of a patient, and to heat said flow of intravenous fluid sufficiently to avoid hyperthermia to the body of a receiving patient.

Additionally, the invention provides a hygienic system for providing and heating a flow of intravenous fluid between a source of intravenous fluid and an intravenous catheter.

The invention further provides a system for accurately administering a flow of intravenous fluid to a patient.

With the present invention is provided a portable unit, in which an electrical energy source for heating of the fluid is provided integrally in the same housing as a pump for controlling the flow of the intravenous fluid by manipulating the outer surface of the intravenous tube. Thus, a simple system for administering a heated intravenous fluid to a under field conditions patient is provided, where the advantages of heating the fluid is combined with the provision of a pump that enables administration of a predefined flow rate of the fluid regardless of vertical position of the container of the intravenous fluid with respect to the patient. When combined with an intravenous tube provided enclosed in a sterile packaging and comprising heating means integrated into the tube for raising the temperature of the intravenous fluid flow, a hygienic system is provided where the sterility of the path of the intravenous fluid flow is easily obtained and preserved, as the intravenous tube can be connected to the container holding the intravenous fluid and to the patient and the portable unit can be connected to the tube for providing heating power and a flow driving pump without interfering with the sterile path of the fluid. Thus, a single infrangible path between the source of intravenous fluid and the intravenous catheter is safely provided.

The present invention relates to a portable unit for providing energy to heat intravenous fluids, the unit comprising a housing wherein is arranged an elongated opening to receive a length of a flexible intravenous tube for connecting a source of intravenous fluid to an intravenous catheter, an energy source comprising one or more rechargeable cells arranged to provide electrical power to heat intravenous fluid flowing in the intravenous tube, and a pump for controlling the flow rate of intravenous fluid in the intravenous tube, the pump operating by manipulating the outer surface of the length of an intravenous tube received in said elongated opening.

The present invention also relates to a system for heating a flow of intravenous fluid comprising the portable unit and an intravenous tube enclosed in a sterile packaging and having a liquid inlet and a liquid outlet for connecting a source of intravenous fluid to an intravenous catheter, said intravenous tube comprising heating means integrated therein for raising the temperature of the intravenous flow in the tube, and connection means for connecting said heating means with an external energy source.

The intravenous tube is preferably delivered sterile and enclosed in a sterile packaging until time of use. Hence, the risk of giving the patient an infection is substantially reduced.

Thus, the present invention relates to the portable unit as well as to a system comprising the portable unit and an intravenous tube including the use hereof.

Hereby, a simple portable system is provided which requires only two connections to external units as compared to most known systems which require multiple connections between several external units, i.e. that a separate heating element is not required by the provision of the system according to present invention to be inserted in between two lengths of intravenous tubing.

The simplicity of the present invention further eases the process of administering intravenous fluids to critical wounded persons and minimises the risk for rescue personnel under stress, e.g. an army medic at the front line or a paramedic at a large scene of accident, form making mistakes during the infusion of heated intravenous fluids, in that a separate heating element is not required to be connected to the flow path from the source of intravenous fluid to the patient.

Further the single infrangible path between a source of intravenous fluid and an intravenous catheter provides a highly hygienic system, because the number of connections wherein the flow of intravenous fluid can be contaminated is limited to a minimum as compared to other systems with multiple connections to external units.

It is advantages that the housing of the portable unit includes mounting means for fastening the unit to a patient receiving the intravenous fluids. In a particular embodiment, the portable unit is formed as a flexible wristband and the mounting means includes straps of the like to fasten the portable unit around the arm of the patient to receive the intravenous fluid.

The energy source is preferably able to supply sufficient power to raise the temperature of the intravenous fluid with an amount between 20° C. and 30° C., preferably between 23° C. and 27° C. at a flow rate of 100 ml/min.

The energy source may be connected to a heating element in the portable unit, which then is in contact with the outer surface of a part of the intravenous tube, preferably the part received in the elongated opening of the portable unit, but in a preferred embodiment of the present invention, the energy source comprises means for supplying electrical power to heating means arranged externally to the unit, such as within the intravenous tube. The means for supplying electrical power are advantageously arranged within or adjacent to the elongated opening of the housing, in particularly so that the electrical contact between said means and corresponding electrical terminals on the tube is established when the intravenous tube is placed correctly in the elongated opening in the portable unit.

In the case where the heating means are integrated into the intravenous tube, it is advantageous that the surface area in contact with the intravenous fluid of said heating means comprises an area of at least 20 cm², preferably at least 30 cm² and most preferred at least 40 cm² so as to ensure that the heating of the fluid is performed gentle. By having a substantially large heating area in contact with the intravenous fluid, a low contact temperature between the heating means and the intravenous fluid is obtained, thereby providing a gentle heating of the intravenous fluid.

In another aspect of the present invention said integrated heating means comprises one or more heating wires inlaid in the enclosure of the intravenous tube. The intravenous fluid is heated while flowing through the enclosure of the intravenous tube, thereby providing a large area of the heating means which is in contact with the intravenous fluid. This means that the intravenous fluid can be gently heated, thus preventing damage to fragile intravenous fluids such as blood.

In yet another aspect of the present invention said integrated heating means comprising heating wires encased in the wall of the intravenous tube. Hence, the heating wires are protected within the wall of the intravenous tube, thereby providing heating means that is resistant for use in harsh environments, such as when the intravenous tube are carried in the field in a densely packed bag which is exposed to large temperature gradients, shocks and blows.

The heating means extends preferably a substantial portion of the tube length preferably between 75 cm and 200 cm of the tube length such as between 125 cm and 175 cm of the tube length. Hence, it is assured that the flow of intravenous fluid is gently heated without being damaged, and that the heat loss through the tube wall is reduced to a minimum.

In a preferred embodiment of the present invention, the heating element extends out from the coupling of the tube to the container for the fluid and into the container, so at to keep the content of the container above freezing temperature during operation and thereby prevent the formation of ice crystals that may damage the fluid, e.g. in case of blood, or may cause clogging of the flow path.

With the pump in the portable unit, a flow of intravenous fluid can be provided without the aid of gravity and the amount of intravenous fluid administered to a patient can easily be controlled by the rescue personnel. The pump may in particular be a peristaltic pump, i.e. a pump operating by manipulating the outer surface of a flexible length of the intravenous tube without having parts of the pump in directly contact with the fluid itself. Thereby, the flow of intravenous fluid delivered to a patient can be controlled without undermining the sterile path of the intravenous fluid from a source of intravenous fluid to a patient. Further the amount of intravenous fluid delivered to the patient can easily be established and controlled. In one embodiment, the intravenous tube comprises two one-way valves and the pump comprises a single piston that during operation alternately compresses and releases the compress of the piece of flexible and elastic length of intravenous tube between the two valves and situated in the elongated opening of the portable unit.

The portable unit may further comprise control means for controlling the operation of at least one element of the unit, in particular the supply of power to the heating of the fluid and/or the pump, in response to at least one control input from means for providing a measure of at least one physical property of the flow of intravenous fluid, such as in particular the temperature of the fluid and/or the flow rate.

In one embodiment, said means for providing a measure of at least one physical property of the intravenous fluid comprises temperature measure means for measuring the temperature of the flow of fluid in the intravenous tube and providing an output accordingly to the control means. The temperature measuring means are preferably arranged within or adjacent to the elongated opening of the housing.

Additionally or alternatively, said means for providing a measure of at least one physical property of the intravenous fluid comprises means for providing a measure of the flow rate of fluid in the intravenous tube and providing an input accordingly to the control means. The means for providing a measure of the flow rate of fluid in the intravenous tube may further comprise alarm means for providing an alarm signal when the source of intravenous fluid is empty and/or comprise alarm means for providing an alarm signal in case the flow of intravenous fluid is interrupted.

It is preferred that the operation of the pump and/or the supply of energy from the energy source to heat intravenous fluid flowing in the intravenous tube are controlled so as to limit the temperature of the flow of intravenous fluid to a temperature interval between 35° C. and 40° C. such as between 36° C. and 38° C.

### BRIEF DESCRIPTION OF THE DRAWINGS

The invention will be described in the following with reference to the drawings of which

FIG. 1 illustrates a system for providing and heating a flow of intravenous fluid according to a preferred embodiment of the present invention, and

FIG. 2a to 2f illustrates cross sectional views of different embodiments of intravenous tubes according to the present invention.

### DETAILED DESCRIPTION OF THE PRESENT INVENTION

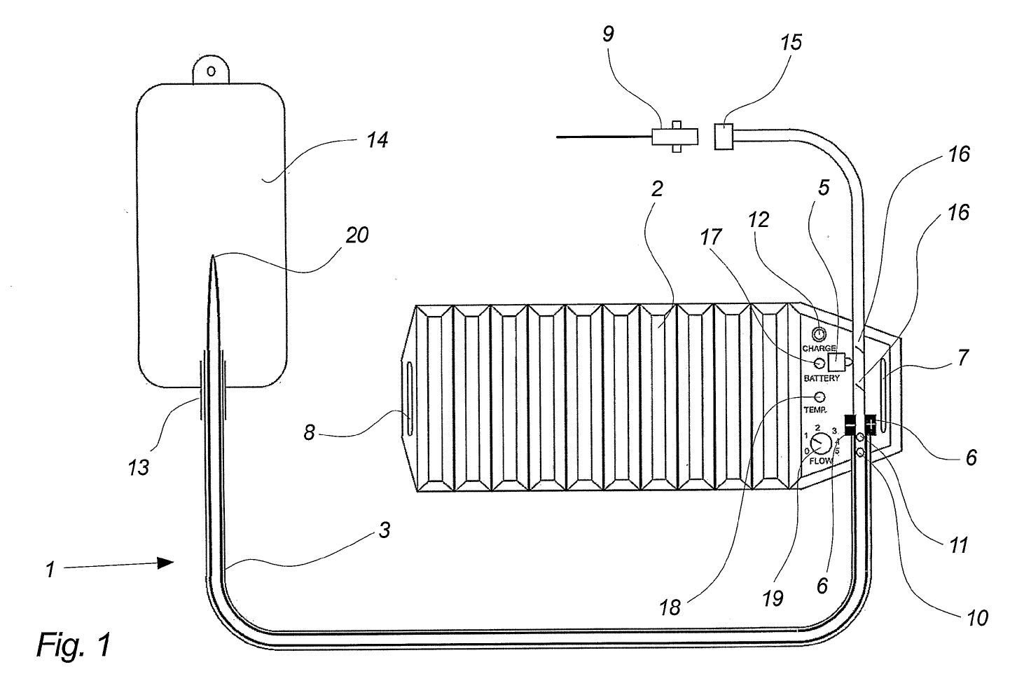

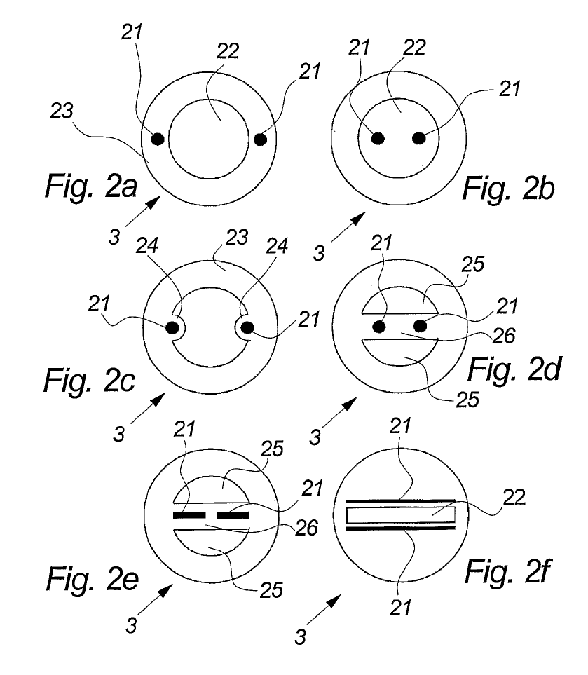

FIG. 1 illustrates a system 1 for heating a flow of intravenous fluid according to a preferred embodiment of the present invention. The system 1 comprises two separate main parts, a portable unit 2 in form of a wristband and an intravenous tube 3 with electrical heating wires (examples shown in FIGS. 2a to 2f) integrated therein. A length of the tube 3 is temporarily arranged in an elongated opening 4 of the wristband 2 when the system is about to be used. The wristband may be used a multitude of times while the intravenous tube 3 is for single use only. A peristaltic pump 5 is integrated in the wristband and operates on the part of the intravenous tube 3 housed in the elongated opening 4 for an accurate control of the intravenous fluid delivered to a patient.

The portable unit 2 comprises an energy source in form of a plurality of rechargeable electrical batteries arranged along the circumference of the wristband. The portable unit 2 further comprises a socket sized to fit a corresponding connector 6 mounted on the intravenous tube 3. By forming the portable unit 2 as a wristband with openings 7, 8 for mounting of a strip to fasten the wristband to an arm of a patient the intravenous tube 3 can be fixated to the wrist of a patient thereby preventing the intravenous catheter 9 from being pulled out of the vein of the patient during transport.

The portable unit 2 further comprises a temperature controller in order to control the heating of the intravenous fluid. Near the connector 6 a built-in temperature sensor 10, e.g. an RTD, provides an input to the temperature controller according to the actual temperature of the intravenous fluid. Hereby, the system is able to prevent that the intravenous fluid is heated above body temperature. As a further safety precaution the portable unit 2 comprises a thermal fuse 11 arranged near the socket 6 in thermal contact with the intravenous fluid e.g. via the connector 6, to cut off the power to the electrical heating elements in case the temperature control system fails. Alternatively, the temperature sensor 10 could also be arranged in the portable unit 2 in thermal contact with the intravenous fluid e.g. via the connector 6. The connector 6 is preferably made of metal or a heat conductive plastic in order to provide the thermal fuse 11 and/or the temperature sensor 10 with an accurate measure of the temperature of the intravenous fluid.

The system is capable of raising the temperature of the intravenous fluid 25° C. above its initial temperature at a flow rate of 100 ml/min. If the temperature of the intravenous fluid reaches body temperature (approximately 37° C.) the temperature controller cuts off the power to the electrical heating wires until the temperature of the intravenous fluid is below body temperature.

The portable unit 2 further comprises a second socket 12 for connecting the unit 2 with a utility grid or the electrical system of a motorised vehicle, thereby providing means for supplying the electrical heating wires with an alternative source of electricity and/or for recharging the rechargeable batteries, when the external unit is connected to an alternative source of electricity.

The intravenous tube 3 comprises a liquid inlet 8 releasably connected to an intravenous bag 14 and a liquid outlet 15 for releasably connecting the intravenous tube 3 to an intravenous catheter 9 to be inserted into a vein of a patient. In order to heat a flow of intravenous fluid flowing through the intravenous tube 3, a pair of electrical heating wires (examples shown in a cross sectional view in FIGS. 2a to 2f) preferably of stainless steel and integrated within the intravenous tube 3 extends inside the tube 3. The electrical heating wires are supplied with electrical power from the portable unit 2 through the connector 6. The heating wires extend 20 into the bag 14 containing the fluid to prevent it from freezing during operation.

A length of intravenous tubing 3 without integrated heating wires extends inside the elongated opening 4 and is equipped with two one-way valves 16, and the peristaltic pump 5 operates on the tube 3 between these valves 16 in order to accurately control the flow of intravenous fluid from the intravenous bag 9 to the intravenous catheter.

The portable unit 2 further comprises a light diode 17 for indication of the charging condition of the rechargeable batteries, a light diode for indication of the fluid temperature being within the correct range and a control knob 19 for manual adjustment of the flow rate.

When the tube 3 is placed in the elongated opening 4 ands the connector 6 of the intravenous 3 tube is inserted into the socket of the portable unit 2, electrical power is automatically transferred from batteries in the portable unit 2 to the electrical heating wires in the intravenous tube 3. The electrical heating wires with an outer diameter of 0.5 millimetres extend approximately 150 cm from the connector 6 to the part 20 extending into the bag 14. Thereby, a gentle heating of the flow of intravenous fluid is assured and damage to fragile intravenous fluids such as blood is prevented.

The peristaltic pump 5, which is also powered by the rechargeable batteries of the portable unit 2, provides a controlled flow of intravenous fluid. Hereby the rescue personnel can administrate the amount of intravenous fluid delivered to a patient according to instructions from a doctor situated at remote location from the scene of accident. The doctor could e.g. be situated at a hospital and send his instructions to the rescue personnel treating a patient in the field via radio or telephone. Additionally the peristaltic pump 5 is capable of measuring the amount of intravenous fluid delivered to a receiving patient, and providing an alarm signal when the source 14 of intravenous fluid is empty. The peristaltic pump 5 further comprises flow measuring means in order to detect if the flow of intravenous fluid is interrupted, thereby providing an alarm signal if the flow of intravenous fluid is interrupted, e.g. if the intravenous tube 3 is twisted or pinched during transport of the patient.

The invention has been exemplified above with reference to specific examples of a system for heating intravenous fluids. However, it should be understood that the invention is not limited to the particular examples described above but may be designed and altered in a multitude of varieties within the scope of the invention as specified in the claims.

FIGS. 2a to 2f illustrates cross sectional views of different embodiments of the intravenous tube 3 with integrated electrical heating wires 11. Although the shape of the electrical heating wires 21 differs in the different embodiments shown in FIGS. 2a to 2f, it is common to all embodiments that the electrical heating wires either extends into the bag 14 of fluid or are short circuited by a metal ring. The embodiments shown in the figures are only meant as examples, so other embodiments with other configurations of the heating wires 21 are also within the scope of the present invention.

FIG. 2a shows a cross sectional view of a first embodiment of the intravenous tube 3 according to the present invention. The intravenous fluid flows through a circular inner portion 22, and is heated via circular electrical heating wires 21 encased within the wall 23 of the intravenous tube 3.

FIG. 2b shows a cross sectional view of a second embodiment of the intravenous tube 3 according to present invention. The intravenous fluid flows through a circular inner portion 22, and is heated via circular heating wires 21 placed directly inside the circular portion 22 of the intravenous tube 3.

FIG. 2c shows a cross sectional view of a third embodiment of the intravenous tube 3 according to present invention. The intravenous fluid flows through the circular inner portion 22 of the intravenous tube 3, and is heated by circular electrical heating wires 21 encased within internal thickenings 24 of the inner portion of the wall 23 of the intravenous tube 3.

FIG. 2d shows a cross sectional view of a fourth embodiment of the intravenous tube 3 according to present invention. The intravenous fluid flows within two semi circular inner portions 25 of the intravenous tube 3 divided by an inner wall 26. The intravenous fluid is heated by circular electrical heating wires 11 encased within the inner wall 26.

FIG. 2e shows a cross sectional view of a fifth embodiment of the intravenous tube 3 according to present invention. The intravenous fluid flows within two semi circular inner portions 25 of the intravenous tube 3 divided by an inner wall 26. The intravenous fluid is heated by rectangular electrical heating wires 21 encased within the inner wall 26. The rectangular shape of the electrical heating wires 21 provides a larger surface area from which heat can be transferred to the intravenous fluid, as compared to the circular electrical heating wires 21.

FIG. 2f shows a cross sectional view of a preferred embodiment of the intravenous tube 3 according to present invention. The intravenous fluid flown through a rectangular inner portion 27 of the intravenous tube, and is heated by rectangular shaped heating wires 21 arranged along the longer sides of the rectangle. The electrical heating wires 21 could advantageously be two sheets of foil. Hereby the intravenous fluid flows past heating wires with a large surface area. Hence, an effective transfer of heat between the heating wires 21 and the intravenous fluid is provided, thereby minimising the heat loss from the intravenous fluid. Further the ratio between the area of the electrical heating element and the power delivered to the heating element can be substantially lowered, thus precluding a meltdown of the heating element. Another advantage of the low power to area ratio is a low contact temperature between the heating wires and the intravenous fluid, thereby providing a gentle heating of the intravenous fluid.

1. A portable unit for providing energy to heat intravenous fluids, the unit comprising a housing comprising:
an elongated opening to receive a length of a flexible intravenous tube for connecting a source of intravenous fluid to an intravenous catheter,
an energy source comprising one or more rechargeable cells arranged to provide electrical power to heat intravenous fluid flowing in the intravenous tube, and
a pump for controlling flow rate of intravenous fluid in the intravenous tube, the pump operating by manipulating an outer surface of the length of an intravenous tube received in said elongated opening by means of a single piston that during operation alternately compresses and releases the compress of the piece of flexible and elastic length of intravenous tube the housing further including mounting means for fastening the unit to a patient receiving the intravenous fluids.

2. (canceled)

3. A portable unit according to claim 1, wherein said energy source is able to supply sufficient power to raise the temperature of the intravenous fluid with an amount between 20° C. and 30° C. at a flow rate of 100 ml/min.

4. A portable unit according to any of claims 1, wherein said energy source comprises means for supplying electrical power to heating means arranged externally to the unit.

5. A portable unit according to claim 4, wherein the said means for supplying electrical power are arranged within or adjacent to the elongated opening of the housing.

6. (canceled)

7. A portable unit according to claim 1, further comprising control means for controlling operation of at least one element of the unit in response to at least one control input from means for providing a measure of at least one physical property of the flow of intravenous fluid.

8. A portable unit according to claim 7, wherein said means for providing a measure of at least one physical property of the intravenous fluid comprises temperature measure means for measuring the temperature of the flow of fluid in the intravenous tube and providing an output accordingly to the control means.

9. A portable unit according to claim 8, wherein the temperature measuring means are arranged within or adjacent to the elongated opening of the housing.

10. A portable unit according to claim 7, wherein said means for providing a measure of at least one physical property of the intravenous fluid comprises means for providing a measure of the flow rate of fluid in the intravenous tube and providing an input accordingly to the control means.

11. A portable unit according to claim 10, wherein said means for providing a measure of the flow rate of fluid in the intravenous tube comprises alarm means for providing an alarm signal when the source of intravenous fluid is empty.

12. A portable unit according to claim 10, wherein said means for providing a measure of the flow rate of fluid in the intravenous tube comprises alarm means for providing an alarm signal in case the flow of intravenous fluid is interrupted.

13. A portable unit according to claim 7 to 12, wherein the control means controls the operation of the pump.

14. A portable unit according to claim 7, wherein the control means controls the supply of power from the energy source to heat intravenous fluid flowing in the intravenous tube.

15. A portable unit according to claim 7, wherein the operation of the pump and/or the supply of energy from the energy source to heat intravenous fluid flowing in the intravenous tube are controlled so as to limit the temperature of the flow of intravenous fluid to a temperature interval between 35° C. and 40° C.

16. A system for heating a flow of intravenous fluid comprising:
an intravenous tube enclosed in a sterile packaging and having a liquid inlet and a liquid outlet for connecting a source of intravenous fluid to an intravenous catheter, said intravenous tube comprising heating means integrated therein for raising the temperature of the intravenous flow in the tube, and connection means for connecting said heating means with an external energy source, and
a portable unit according to claim 1, wherein said energy source comprises an external energy source.

17. A system according to claim 16, wherein the surface area in contact with the intravenous fluid of said heating means comprises an area of at least 20 cm².

18. A system according to claim 16, wherein said integrated heating means comprises one or more heating wires inlaid in the enclosure of the intravenous tube.

19. A system according to claim 16, wherein said integrated heating means comprising heating wires encased in the wall of the intravenous tube.

20. A system according to claim 16, wherein said heating means extends a substantial portion of the tube length comprising between about 75 cm and about 200 cm of the tube length.

21. A system according to 16 configured for heating a flow of intravenous fluid from a source of intravenous fluid to a patient.

22. A system according to claim 16, wherein the intravenous tube comprises two one-way valves and the single piston of the pump compresses and releases the compress of the piece of flexible and elastic length of intravenous tube between the two valves.

23. A system according to claim 16, wherein the energy source is connected to a heating element in the portable unit, which then is in contact with the outer surface of a part of the intravenous tube.

24. A system according to claim 23, wherein the heating element in the portable unit during operation is in contact with the outer surface of part of the intravenous tube received in the elongated opening of the portable unit.
